(19) United States
(12) Patent Application Publication
(10) Pub. No.: US 2006/0035950 A1
(43) Pub. Date: Feb. 16, 2006

(54) Novel processes for preparing substantially pure anastrozole

(76) Inventors: Mohammed Alnabari, Hura (IL); Boris Freger, Beer-Sheva (IL); Oded Arad, Rechovot (IL); Lior Zelikovitch, Mazkeret Batia (IL); Yana Seryi, Beer-Sheva (IL); Edna Danon, Meitar (IL); Guy Davidi, Even-Yehuda (IL); Joseph Kaspi, Givatayim (IL)

Correspondence Address: Martin MOYNIHAN;c/o ANTHONY CASTORINA, SUITE 207, 2001 JEFFERSON DAVIS HIGHWAY, ARLINGTON, VA, 22202, US

(21) Appl. No.: 11/199,226

(22) Filed: Aug. 9, 2005

Related U.S. Application Data
(60) Provisional application No. 60/599,546, filed on Aug. 9, 2004.
(60) Provisional application No. 60/599,581, filed on Aug. 9, 2004.

(51) Int. Cl. C07D 249/08; A61K 31/4196

(52) U.S. Cl. 514/383; 548/262.2

(57) ABSTRACT

The present invention provides novel processes for purifying anastrozole, devoid of using liquid chromatography. The purification processes are via the isolated anastrozole salt forms, either by crystallization or by selective acidic extractions, and optionally in both cases, converting the purified anastrozole salt to anastrozole base. Also provided is an improved process for the synthesis of anastrozole, which is obtained by alkylating the isolated and purified starting material 3,5-bis(2-cyanoprop-2-yl)benzylbromide, the process being devoid of using toxic, hazardous and environmental unfriendly solvents and reagents.

### RELATED APPLICATIONS

The present application claims priority from U.S. Provisional Patent Application No. 60/599,546 and U.S. Provisional Patent Application No. 60/599,581, both filed on Aug. 9, 2004, the contents of which are incorporated herein by reference.

### FIELD OF THE INVENTION

The present invention provides novel processes for preparing substantially pure anastrozole devoid of using liquid chromatography and hazardous solvents and reagents. Instead, complementary purification techniques are used for obtaining substantially pure anastrozole.

### BACKGROUND OF THE INVENTION

Anastrozole is a common name of the chemically known substance 2,2′-[5-(1H-1,2,4-triazol-1-ylmethyl)-1,3-phenylene]di(2-methylpropionitrile), which is also described as α,α,α′,α′-tetramethyl-5-(1H-1,2,4-triazol-ylmethyl)-1,3-benzenediaceto nitrile, and is represented by formula (I):

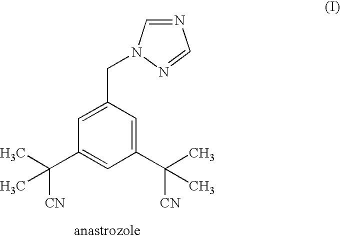

Anastrozole is a selective and potent non-steroidal drug which inhibits the action of the enzyme aromatase. It is used for the treatment of advanced breast cancer in postmenopausal women with disease progression following tamoxifen therapy. Anastrozole is further recognized and granted for treatment of postmenopausal women with hormone receptor positive or hormone receptor unknown, locally advanced or metastatic breast cancer and also for adjuvant treatment of postmenopausal women with hormone receptor positive early breast cancer.

The growth of tumors in many breast cancers can be stimulated by estrogen. In postmenopausal women, the principal source of circulating estrogen is the conversion of adrenally-generated androstenedione to estrone by the enzyme aromatase. Inhibition of aromatase affects the aromatization of the ring A in the metabolite formation of various steroid hormones. Anastrozole has been shown to inhibit this in vivo aromatization by 96-97% and to suppress plasma estrogen levels by up to 94%.

The synthesis of anastrozole is described in U.S. Pat. Nos. 4,935,437 and RE 36617 (a re-issue of U.S. Pat. No. 4,935,437 assigned to AstraZeneca Pharmaceuticals), which are incorporated herein by reference. These patents describe two synthetic routes for preparing anastrozole, one starting from methyl 3,5-dimethylbenzoate in a six-step process and the other from 3,5-bis(bromomethyl)toluene in a three-step process. The second process is preferable because it is much shorter and easier to perform, however both processes involve a benzylic bromination stage with N-bromosuccinimide (NBS) in CCl₄.

The first process is described in Scheme 1 below. Bromination of methyl 3,5-dimethylbenzoate with N-bromosuccinimide (NBS) in CCl₄ affords a 3,5-bis(bromomethyl) compound, which is subsequently treated with potassium cyanide to afford a dinitrile. The dinitrile is alkylated, then reduced to the corresponding alcohol. The alcohol is converted to an alkyl chloride intermediate, and anastrozole is then obtained by reaction of the latter compound with sodium triazole.

The final product is purified by flash column chromatography, using a repeated elution with a methanol:chloroform solvent mixture.

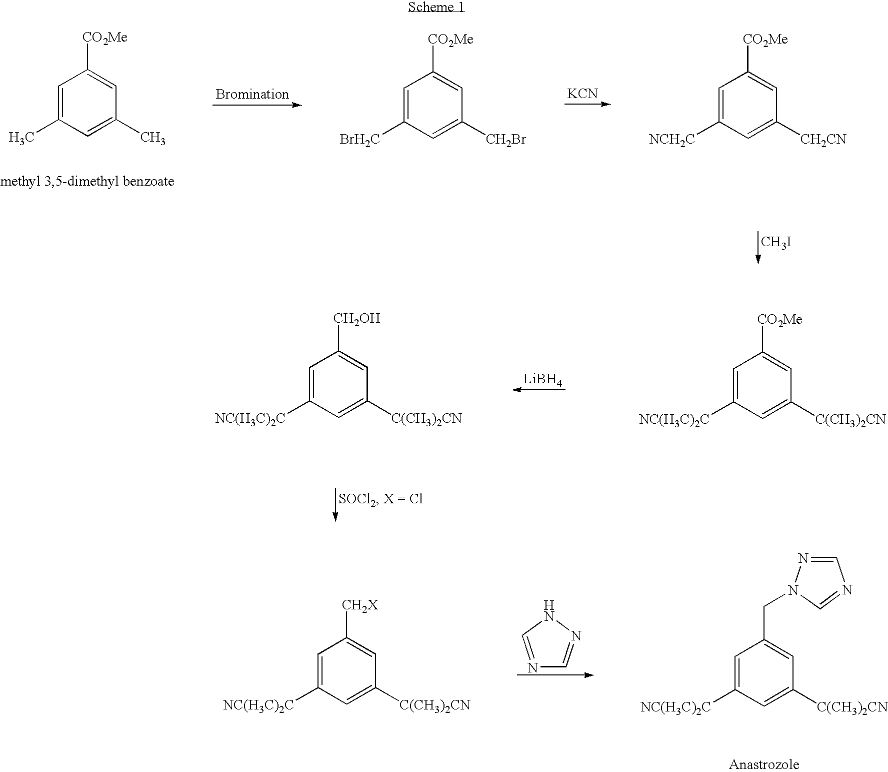

In the second process, described in Scheme 2, the starting material, 3,5-bis(bromomethyl)-toluene, is reacted with potassium cyanide in dichloromethane in the presence of a catalytic amount of tetrabutylammonium bromide (TBAB) to obtain 2,2′-(5-methyl-1,3-phenylene)diacetonitrile. The product is mixed with iodomethane and sodium hydride in DMF to thereby obtain 2,2′-(5-methyl-1,3-phenylene) di(2-methylpropionitrile), (also referred to as 3,5-bis(2-cyanoprop-2-yl)toluene).

The next step in this process includes bromination of 2,2′-(5-methyl-1,3-phenylene)di(2-methyl-propionitrile), which is performed according to the traditional procedure, by adding benzoyl peroxide to a mixture of N-bromosuccinimide (NBS) and the substrate, in 1:1 molar ratio or more, in carbon tetrachloride. The mixture is typically refluxed for 2 hours, cooled, filtered, and the filtrate is evaporated to dryness under reduced pressure. The residue obtained is dissolved in DMF and sodium triazole is added. After completion of the reaction, anastrozole is purified by flash column chromatography, eluting with ethyl acetate.

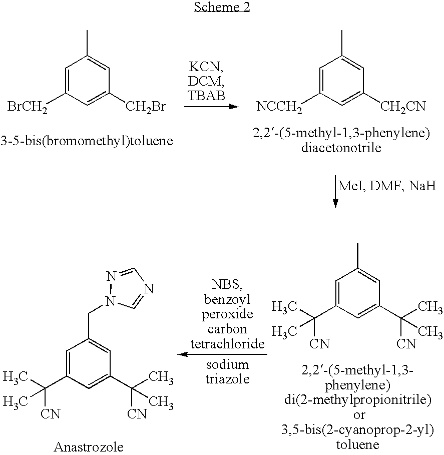

Thus, the bromomethyl intermediate in the second process is not isolated, but is directly converted to anastrozole in situ. As a result of using the non-isolated intermediate, an impure final product is obtained. In both processes, crude anastrozole is purified by tedious, inefficient and expensive chromatographic methods.

Furthermore the chromatographic use of solvents such as methanol and chloroform (the latter being a carcinogenic solvent) is disadvantageous with respect to industrial application.

The well-known benzylic bromination stage, which is traditionally performed in CCl₄, has been studied by several research groups. Goldberg et al., J. Org. Chem. 1992, 57, 6374, for example, have focused on conditions for improving the side chain (benzylic) bromination of methyl anisoles. No reference was made to ring bromination.

Gruter et al., J. Org. Chem. 1994, 59, 4473, have described the predominant benzylic bromination with NBS in CCl₄, which was observed for methyl substituted anisoles.

Mitchell et al, J. Org. Chem., 1979, 44, 4733, have reported that by using a polar solvent such as DMF instead of CCl₄, ring bromination took place, instead of side chain bromination.

Ross et al. J. Amer. Chem. Soc., 1958, 80, 4327, have described the use of a polar solvent for enhancement of reactivity of NBS. However, competitive bromination of the solvent substantially decreased the reaction yields.

Carreno et al., J. Org. Chem. 1995, 60, 5328-5331, have discovered that by brominating anisole derivatives such as: 2,3 and 4-methylanisole, as well as 2,3 and 2,6-dimethylanisole with NBS in CCl₄, the sole preference was for side chain (benzylic) bromination. By using acetonitrile as solvent, instead of CCl₄, ring bromination products were the only detected products.

The explanation that was given to these findings was that the reaction with NBS/CCl₄ proceeds by free radical mechanism, while ring bromination takes place by the ionic mechanism involved in electrophilic substitution.

Because of the difficulties concerned with using carbon tetrachloride and/or benzoyl peroxide on industrial scale, it would be highly desirable to develop a process for preparing anastrozole, devoid of using hazardous solvents and reagents (such as CCl₄ and benzoyl peroxide), which will enable obtaining purified anastrozole without using liquid chromatography.

### BRIEF SUMMARY OF THE INVENTION

The present invention provides a novel process for preparing substantially pure anastrozole, the process comprising the steps of:

preparing the bromo intermediate 3,5-bis(2-cyanoprop-2-yl)benzylbromide;

isolating the said bromo intermediate;

optionally purifying the bromo intermediate by crystallization or precipitation;

converting the isolated intermediate to anastrozole; and

purifying the anastrozole thus obtained by crystallization and/or by selective acidic extraction of anastrozole's isomer from an organic solution of crude anastrozole.

According to the present invention, the process for preparing anastrozole, which is depicted in Scheme 3, uses the starting material 3,5-bis(2-cyanoprop2-yl)toluene, which is brominated to the benzyl bromide intermediate 3,5-bis(2-cyanoprop-2-yl)benzylbromide in an organic solvent belonging to class 3 or class 2. 3,5-bis(2-cyanoprop-2-yl)benzylbromide is purified by crystallization or precipitation and subsequently alkylated with sodium triazole, or triazole under basic conditions, in an organic solvent belonging to class 3 or class 2. Hence, the usage of carbon tetrachloride is avoided both in the bromination and alkylation steps. Finally, the crude anastrozole is purified, as described in detail herein, without using liquid chromatography, to obtain a substantially pure product.

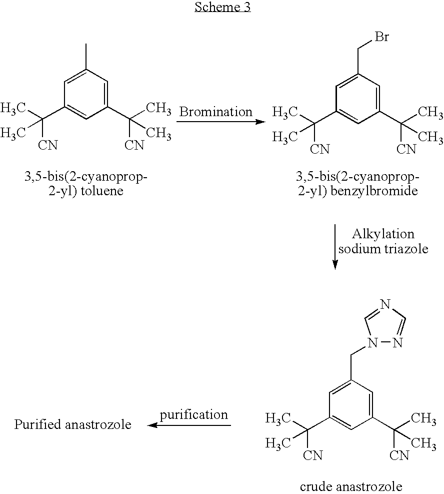

### DESCRIPTION OF THE PREFERRED EMBODIMENTS

The present invention is predicated on the surprising and unexpected discovery, which is contrary to the teaching of the U.S. Pat. Nos. 4,935,437 and RE 36,617 patents, that it is unnecessary to use column chromatography for obtaining substantially pure anastrozole. According to the present invention, anastrozole may be purified via its isolated salt form, such as the hydrochloride or hydrobromide salt, and optionally further purified using different purification techniques, for example, by crystallization. The said purification method generally comprises the steps of:

precipitating anastrozole salt (such as hydrochloride or hydrobromide salts) by acidification; and

crystallizing the precipitated anastrozole salt from an organic solvent or a mixture of organic solvents.

According to one embodiment of the present invention, a suitable crystallization solvent is selected from the group consisting of C₁-C₆ alcohols, wherein preferred alcohols are: methanol, ethanol, n-propanol, isopropyl alcohol, n-butanol, sec-butanol, R₁COOR₂ esters while R₁═C₁-C₅ alkyl and R₂═C₁-C₅ alkyl, wherein preferred esters are: ethyl acetate, isopropyl acetate, n-butyl acetate, and isobutyl acetate, acetonitrile, C₁-C₆ ketones, wherein preferred ketones are: acetone, methylethyl ketone, diethyl ketone, methylpropyl ketone, and methylisobutyl ketone, R₁OR₂ ethers while R₁═C₂-C₅ alkyl and R₂═C₂-C₁ alkyl, wherein preferred ethers are: diethyl ether, diisopropyl ether, methyl tert-butyl ether and THF, DMF, toluene, xylenes, water or any mixture thereof. More preferably, a suitable solvent for crystallization is selected from the group consisting of toluene, isopropyl alcohol, ethanol, ethyl acetate, acetone, water or mixture thereof.

According to another embodiment of the present invention acidification may be performed by bubbling gaseous inorganic acid such as hydrogen chloride or hydrogen bromide or by adding an aqueous solution of inorganic acid to the organic solution of crude anastrozole. Preferably the inorganic acid is sulfuric acid, hydrochloric acid or hydrobromic acid and more preferably it is hydrochloric acid.

According to yet another embodiment of the present invention the purified salt thus obtained may be subsequently converted to the base form by neutralization with an inorganic basic solution.

According to one aspect of the present invention, the basic solution is prepared by dissolving an inorganic base in water. The inorganic base may be selected from the group consisting of sodium hydroxide, potassium hydroxide, potassium carbonate potassium bicarbonate, sodium carbonate, and sodium bicarbonate. The preferred inorganic base is sodium carbonate.

According to yet another aspect of the present invention the inorganic basic solution is a concentrated sodium carbonate solution. Anastrozole base may be extracted with an organic solvent such as ethyl acetate or toluene, preferably toluene, and then conveniently isolated by filtration of the crystals after evaporating the solvent.

The present invention provides a novel process for purifying anastrozole by crystallization comprising the steps of:

a) dissolving crude anastrozole in a suitable organic solvent;

b) adding an inorganic acid to thereby form an anastrozole salt;

c) crystallizing said anastrozole salt, to thereby obtain anastrozole crystals;

d) filtering off said crystals and washing with said organic solvent; and

e) optionally re-crystallizing said anastrozole salt from an organic solvent.

According to an aspect of the present invention, the purification of anastrozole salt via crystallization from a suitable organic solvent does not yield a product that conforms to pharmaceutical requirements without further purification, although crystallization is effective in removing most of the impurities.

According to the present invention, a possible explanation to this phenomenon (that pharmaceutically pure anastrozole cannot be obtained by crystallization without further purification) is that one of the main impurities in the synthesis of anastrozole is its isomer 2,2′-[5-(4H-1,2,4-triazol-1-ylmethyl)-1,3-phenylene]di(2-methyl-propionitrile) (hereinafter isoanastrozole, II).

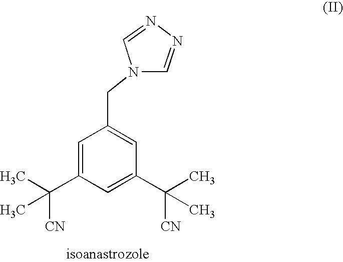

This isomer cannot be separated from anastrozole by precipitation or crystallization because the solubility properties of the two isomers are very similar.

The term “further purification”, in the context of the present invention, means using filtration through packed-bed silica or, optionally and preferably, using selective extraction with suitable acidic solution, having a pH range of 0.7-1.7.

According to the present invention crystallization enables eliminating almost all of the so-called hydrophobic impurities (organic molecules that do not contain acidic or basic functional groups), which are soluble in the crystallization solvent.

Table 1 provides the results obtained by crystallizing anastrozole hydrochloride from different solvents or solvent mixtures.

TABLE 1

| | | | | Total other |
|---|---|---|---|---|
| Solvent or | | anastrozole | isoanastrozole | impurities |
| solvent | | % peak area | % peak area by | % peak area by |
| mixture | Yield, % | by HPLC | HPLC | HPLC |
| toluene | 71.7 | 95.8 | 3.3 | 0.9 |
| ethanol | 75 | 92.8 | 1.5 | 5.7 |
| isopropyl | 84 | 93.7 | 1.1 | 5.2 |
| alcohol | | | | |
| ethyl acetate | 73 | 89.5 | 2.2 | 8.3 |
| toluene and | 82 | 88.5 | 8.1 | 3.4 |
| isopropyl | | | | |
| alcohol | | | | |
| acetone and | 60 | 95.4 | 1.5 | 3.1 |
| water | | | | |

A preferred embodiment of the present invention is the surprising finding that by selectively extracting a solution of crude anastrozole in a mixture of organic solvent and a suitable acidic solution, at a concentration of about 1N, having a pH range of 0.7-1.7, almost all of the isoanastrozole is removed and a substantially pure product is obtained after precipitation of anastrozole salt with gaseous hydrogen chloride (see Scheme 4).

According to one aspect of the present invention, the pH range of 0.7-1.7 was determined by selecting the optimal acidic extracting conditions from various experiments of which, extracting the reaction mixture (containing anastrozole and isoanastrozole) was carried out with acidic solutions having different pH values.

The content of anastrozole and residual isoanastrozole (after the selective extraction) in the organic solution were measured by HPLC. The data is presented in Table 2. A certain quantity of anastrozole is also extracted along with isoanastrozole at each pII value to the aqueous acidic solution, thus leading to a decrease in the yield of the obtained crude anastrozole. Therefore the optimal pH value is the best compromise between the minimal loss of anastrozole along with low content of isoanastrozole in the organic solution.

TABLE 2

| | | Content of | | |
|---|---|---|---|---|
| | pH of | anastrozole | Content of | Crude |
| | acidic | in toluene | isoanastrozole in | anastrozole |
| | solution | (by HPLC) % | toluene (by HPLC) % | yield % |
| 1 | 2.22 | 95.50 | 4.50 | 75 |
| 2 | 1.68 | 96.23 | 3.77 | 65 |
| 3 | 1.51 | 98.98 | 1.02 | 72 |
| 4 | 1.27 | 99.0 | 1.0 | 82 |
| 5 | 1.14 | 99.40 | 0.60 | 77 |
| 6 | 1.06 | 99.66 | 0.34 | 68 |
| 7 | 0.98 | 99.75 | 0.25 | 65 |

According to one embodiment of the present invention, substantially pure anastrozole having purity greater than 99.5% and even greater than 99.7% (by HPLC) is obtained, after extracting the solution of crude anastrozole, by converting the purified anastrozole to anastrozole salt, and subsequently neutralizing the solution of purified anastrozole salt and precipitating free anastrozole from cyclohexane.

According to the present invention it is therefore not needed to use laborious and expensive liquid chromatography purification for obtaining substantially pure anastrozole. The explanation to this surprising finding might be that the ionization constants of anastrozole and isoanastrozole are not identical because of the different molecular structures of the two isomers, hence their pKa values are different. As a result, anastrozole may be separated from almost all the isoanastrozole impurity content by acidic extraction. The acid extraction process does not eliminate the so-called hydrophobic impurities because these molecules do not contain nitrogen atoms. The so-called hydrophobic impurities are soluble in the crystallization solvent, therefore it is possible to remove almost all of them already after the anastrozole salt precipitation stage.

According to the present invention a typical purification procedure comprises dissolving crude anastrozole in an organic solvent followed by selective extraction with an aqueous acidic solution having pH range of 0.7-1.7, followed by repeated extractions (two or more) with a fresh portion of the same acidic solution. After phase separations, the organic solution is saturated with inorganic acid and precipitation occurs, the salt formed is washed in the same solvent, filtered and dissolved in a mixture of a second organic solvent, water and an inorganic base. Addition of cyclohexane to the thus obtained solution of anastrozole base results in the precipitation of pure anastrozole having a pharmaceutical quality, (content of isoanastrozole is less than 0.1% and even less than 0.05% by HPLC).

According to one embodiment of the present invention, the acidic aqueous solutions, which arc preferably, but not limited to, buffer solutions, arc prepared by mixing an acid, preferably but not limited to an inorganic acid, optionally with a salt of the said inorganic acid, either in anhydrous form or in hydrated form, or any combinations of acids and salts thereof, to form an acidic aqueous solution, having specific pH range of 0.7-1.7, preferably 1.0-1.4 and more preferably at about 1.2.

According to another embodiment of the present invention the said acidic aqueous solutions may be prepared by mixing an inorganic acid selected from the group consisting of phosphoric acid, sulfurous acid, sodium hydrogen sulfate, sulfuric acid, preferably sulfuric acid, with a salt selected from the group consisting of monosodium phosphate, monopotassium phosphate, monoammonium phosphate, sodium sulfate, potassium sulfate, magnesium sulfate and ammonium sulfate, preferably sodium sulfate.

According to another embodiment of the present invention a suitable organic solvent for dissolving crude anastrozole is any water-immiscible solvent, selected from the group consisting of dichloromethane, ethyl acetate, isopropyl acetate, n-butyl acetate, isobutyl acetate, diethyl ether, diisopropyl ether, methyl tert-butyl ether, xylenes and toluene or a mixture thereof. Preferably the solvent is toluene.

According to yet another embodiment of the present invention, the organic solution is saturated, prior to obtaining the anastrozole base, either with liquid inorganic acid such as sulfuric acid, hydrochloric acid or hydrobromic acid, preferably hydrochloric acid, or with gaseous mineral acid, to thereby obtain a salt as a precipitate.

According to yet another embodiment of the present invention, the suitable gaseous mineral acid is selected from hydrogen bromide and hydrogen chloride, and preferably the gaseous mineral acid is hydrogen chloride.

According to yet another embodiment of the present invention, the suitable second organic solvent for re-dissolving the precipitated anastrozole, prior to the mixing with inorganic base, is any water-immiscible solvent, selected from the group consisting of dichloromethane, ethyl acetate, isopropyl acetate, n-butyl acetate, isobutyl acetate, diethyl ether, diisopropyl ether, methyl tert-butyl ether, xylenes and toluene. Preferably the solvent is ethyl acetate.

According to yet another embodiment of the present invention, the inorganic base is selected from the group consisting of sodium hydroxide, potassium hydroxide, sodium carbonate, potassium carbonate, sodium bicarbonate, and potassium bicarbonate. Preferably the inorganic base is sodium carbonate.

The present invention provides a novel process for purifying anastrozole, using selective extraction with an aqueous acidic solution comprising the steps of:

a) dissolving impure anastrozole in a suitable organic solvent;

b) adding aqueous acidic solution having a pH in a range of 0.7-1.7, mixing, selectively extracting and subsequently re-extracting and phase separating;

c) acidifying the organic phase and obtaining anastrozole salt as crystals thereof;

d) filtering the crystals off and washing with said organic solvent;

e) optionally suspending the crystals in a second organic solvent and converting said anastrozole salt to anastrozole base;

f) optionally partially evaporating said second organic solvent; and

g) optionally precipitating said anastrozole base by adding a suitable hydrophobic organic solvent.

Scheme 4 describes the anastrozole purification processes via an anastrozole salt.

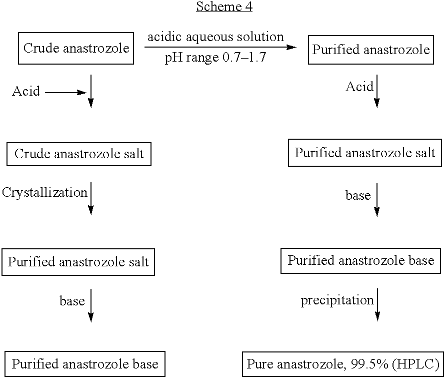

According to the present invention, the purification of anastrozole, as described herein, consists of a fast, simple and high-yield method, performed in mild conditions. The purification techniques described herein are complementary for purification of anastrozole, being able to eliminate isoanastrozole as well as the so-called hydrophobic impurities. The process may be readily up-scaled to industrial production, being devoid of the disadvantages of the previous known processes, associated with the technically difficult flash chromatography. Furthermore, since the alkylation, described in Scheme 3, is not carried out in carbon tetrachloride and other organic solvents that belong to class 3 or class 2 are used instead, the safety problems encountered while using carbon tetrachloride are eliminated.

Another advantage of the present invention is that the starting material is the isolated intermediate: 3,5-bis(2-cyanoprop-2-yl)benzylbromide, which may be purified by crystallization, hence enabling a straightforward and more facile purification because said crystallization significantly diminish the quantity of impurities prior to anastrozole purification.

The following is a summary of the advantages of the purification process:

1. Anastrozole salt may be readily obtained by either acidifying the organic reaction phase with aqueous mineral acid solutions such as hydrochloric acid or by bubbling gaseous hydrogen bromide or gaseous hydrogen chloride, preferably gaseous hydrogen chloride;

2. Substantially pure anastrozole, having a purity over 99.5% and even over 99.7% (by HPLC), may be prepared by repetitive selective acidic extractions, thus anastrozole purification may be carried out without using column chromatography;

3. The process for preparing a substantially pure anastrozole may be based on the isolated purified starting material 3,5-bis(2-cyanoprop-2-yl)benzylbromide, which enables straightforward and more facile purification;

4. The purified anastrozole salt may be conveniently converted to the base form by using an inorganic basic solution, wherein the base is selected from the group consisting of sodium hydroxide, potassium hydroxide, potassium carbonate, potassium bicarbonate, sodium carbonate, and sodium bicarbonate, preferably sodium carbonate; and

5. The purified anastrozole base may be selectively extracted from the aqueous medium with an organic solvent such as toluene or ethyl acetate then conveniently further purified by precipitation.

According to another preferred embodiment of the present invention, an improved process is provided for preparing anastrozole via the benzyl bromide intermediate as illustrated in detail in Scheme 3.

According to one embodiment of the present invention, crude anastrozole is obtained by alkylating 3,5-bis(2-cyanoprop-2-yl)benzylbromide with sodium triazole or with triazole, both in basic conditions. Preferably the alkylation reaction is carried out using sodium triazole and potassium carbonate, or triazole and potassium carbonate, or triazole and lithium tert-butoxide in an organic solvent or mixture of organic solvents.

According to another embodiment of the present invention, the reaction solvent is selected from the group consisting of C₁-C₆ alcohols, wherein preferred alcohols are: methanol, ethanol, n-propanol, isopropyl alcohol, n-butanol, sec-butanol, R₁OR₂ ethers while R₁═C₂-C₅ alkyl and R₂═C₂-C₅ alkyl, wherein preferred ethers are: diethyl ether, diisopropyl ether, methyl tert-butyl ether, and THF, acetonitrile, polar solvents wherein preferred polar solvents are: DMF, DMA, DMSO, and NMP, or mixture thereof. More preferably, the solvent is selected from the group consisting of DMF and isopropyl alcohol or mixture thereof.

The present invention provides an improved process for preparing anastrozole, comprising the steps of:

a) dissolving 3,5-bis(2-cyanoprop-2-yl)benzylbromide in an organic solvent other than carbon tetrachloride that belong to class 3 or class 2;

b) reacting 3,5-bis(2-cyanoprop-2-yl)benzylbromide with sodium thiazole or triazole under basic conditions; and

c) purifying the obtained anastrozole essentially as described herein.

As is described in the Background Section of the present invention, the presently known processes for preparing anastrozole involve benzylic bromination with N-bromo succinimide of toluene derivatives such as 3,5-bis(2-cyanoprop2-yl) toluene, as the final synthesis step. This reaction is typically carried out in carbon tetrachloride as reaction solvent.

According to the industrial guideline on residual solvents, last issued on 1997, (Appendixes 5-7: toxicological data for class 1-3 solvents respectively), published by the International Conference on Harmonization (ICH), the use of industrial solvents is restricted according to their safety features. The industrial solvents are divided into three main classes:

Class 1: Solvents that should not be employed in the manufacture of drug substances or drug products because of their unacceptable toxicity or their deleterious environmental effect. Solvents that belong to this class are: benzene, carbon tetrachloride, 1,2-dichloroethane and others.

Class 2: Solvents that should be limited in pharmaceutical products because of their inherent toxicity. Important industrial solvents that belong to this class are chlorinated solvents such as dichloromethane, hydrocarbons such as hexane and aromatic solvents such as toluene.

Class 3: Solvents that may be regarded as less toxic and of lower risk to human health. Important industrial solvents that belong to this class are, ketones, esters alcohols and others.

Carbon tetrachloride is a toxic and suspect carcinogen class 1 solvent, and therefore the use of this solvent as a medium for industrial application is highly disadvantageous and very problematic.

In addition, benzoyl peroxide, which is used in the second process described above (see, scheme 2), is known as a toxic, carcinogenic reagent that is susceptible to produce explosions, thus rendering its industrial use highly disadvantageous and problematic.

According to the present invention the key intermediate in the synthesis of anastrozole, 3,5-bis(cyanoprop-2-yl)benzylbromide, is obtained by benzylic bromination of the starting material 3,5-bis(2-cyanoprop-2-yl)toluene, as illustrated in Scheme 3, in an organic solvent belonging to class 3 or class 2.

The present invention provides an improved process for preparing 3,5-bis(2-cyanoprop-2-yl)benzylbromide comprising the steps of:

a) dissolving 3,5-bis(2-cyanoprop-2-yl)toluene in an organic solvent, which belongs to class 3 or class 2;

b) adding a brominating agent optionally followed by adding benzoyl peroxide;

c) heating the mixture obtained in step b) under reflux for at least about 4 hours, then cooling to room temperature;

d) filtering out solids obtained in step c);

e) washing the remaining organic solution obtained in step d) first with water then with an inorganic basic solution and a slightly acidic solution;

f) separating the phases formed in step e) and drying the organic phase obtained over magnesium sulfate;

g) evaporating the solvent, optionally under reduced pressure, to thereby produce 3,5-bis(2-cyanoprop-2-yl)benzylbromide; and

h) optionally crystallizing 3,5-bis(2-cyanoprop2-yl)benzylbromide from an organic solvent.

According to one embodiment of the present invention the intermediate 3,5-bis(cyanoprop-2-yl)benzylbromide may be purified by crystallization or precipitation and subsequently alkylated with sodium triazole, or triazole under basic conditions, in an organic solvent also belonging to class 3 or class 2. Hence, the usage of carbon tetrachloride is avoided both in the bromination and alkylation steps.

According to the present invention, in a search for an environmentally friendly solvent that can efficiently replace the use of carbon tetrachloride, it has surprisingly been discovered that the bromination reaction described above may be carried out in a medium that is safer and more environmentally friendly than CCl₄, while further avoiding the use of the hazardous reagent benzoyl peroxide.

According to a preferred embodiment of the present invention, a suitable class 3 reaction solvent is selected from the group consisting of R₁COOR₂ esters, while R₁═C₁-C₅ alkyl and R₂═C₁-C₅ alkyl, wherein preferred esters are: methyl acetate, ethyl acetate, n-propyl acetate, isopropyl acetate, n-butyl acetate, isobutyl acetate, and tert-butyl acetate, C₁-C₆ ketones, wherein preferred ketones are: acetone, methylethyl ketone, diethyl ketone, methylpropyl ketone, and methylisobutyl ketone. Suitable class 2 reaction solvent is selected from the group consisting of polar solvents, wherein preferred polar solvents are: DMSO, DMF, DMA, and NMP, halogenated solvents, wherein preferred halogenated solvent is dichloromethane, THF, acetonitrile, and isopropyl acetoacetate.

The use of benzoyl peroxide may be avoided by either:

using UV light with reflux, in dichloromethane, for about 4 hour or optionally for longer time, or

conducting the reaction in acetonitrile.

Therefore, according to the present invention, the more preferred solvents are acetonitrile and dichloromethane.

According to the present invention, not all the solvents in class 3 and 2 produce equally good results. While screening some of the above mentioned solvents it was found that in the presence of some solvents, the purity of the product was not high, either because the solvent was susceptible to bromination or because the content of the by-product 3,5-bis(cyanoprop-2-yl)benzal bromide (see formula (III) below) was too high.

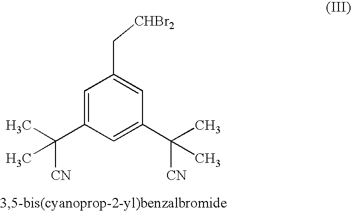

Table 3 provides the solvent screening results concerning the preparation of 3,5-bis(cyanoprop-2-yl)benzyl bromide in various reaction solvents.

TABLE 3

| | | Content of |
|---|---|---|
| | | the benzyl |
| | | bromide |
| | | intermediate |
| | | in reaction |
| | | mixture |
| Solvent | Reaction conditions | (HPLC) |
| Dichloromethane | benzoyl peroxide was not used in the | 87.2% |
| | reaction. UV light was used | |
| Methyl acetate | benzoyl peroxide was used in the | 47.2% |
| | reaction. UV light was not used | |
| Ethyl acetate | benzoyl peroxide was used in the | 51.0% |
| | reaction. UV light was not used | |
| Isopropyl acetate | benzoyl peroxide was used in the | 68.9% |
| | reaction. UV light was not used | |
| Isopropyl | benzoyl peroxide was used in the | 47.7% |
| acetoacetate | reaction. UV light was not used | |
| tert-butyl acetate | benzoyl peroxide was used in the | 61.8% |
| | reaction. UV light was not used | |
| Acetonitrile | benzoyl peroxide was not used in the | 80.6% |
| | reaction. UV light was not used | |
| Acetone | benzoyl peroxide was used in the | 72.1% |
| | reaction. UV light was not used | |

According to the present invention, longer reaction times in dichloromethane cause increased levels of the impurity 3,5-bis(cyanoprop-2-yl)benzalbromide. Therefore, an optimal reaction time is the best compromise between minimal loss of yield due to accumulation of 3,5-bis(cyanoprop-2-yl)benzalbromide in the reaction mixture and insufficient yield of 3,5-bis(cyanoprop-2-yl)benzylbromide. In practice, it is preferable to reflux the dichloromethane solution for about 4 hours, thus a crude product is obtained in more than 85% yield (example 13). If longer times are used, the content of the starting material 3,5-bis(2-cyanoprop-2-yl)toluene may be reduced to less than 2.5%, but on the other hand, the content of 3,5-bis(cyanoprop2-yl)benzalbromide is increased, leading to lower product yield (example 14). However 3,5-bis(cyanoprop-2-yl)benzalbromide may be easily removed by crystallization from a polar solvent.

In a preferred embodiment of the present invention the most preferable solvent for carrying out the reaction of bromination is dichloromethane, being a water immiscible solvent that enables simple work-up procedure of the reaction mixture. Acetonitrile, which is a water-miscible solvent, cannot be used in the working-up stage, which includes washing with aqueous solutions, that can form an emulsion or uniform solution with an acetonitrile solution, and should be therefore removed before such a stage.

In another embodiment of the present invention purified crystalline 3,5-bis(cyanoprop-2-yl)benzylbromide is obtained by subsequent washings of the reaction mixture first with water, followed by inorganic basic solution and finally with mild acidic solution.

In yet another embodiment of the present invention, the suitable inorganic basic solutions include, for example, aqueous solutions of sodium carbonate, potassium carbonate, potassium hydroxide and sodium hydroxide, preferably a sodium hydroxide aqueous solution. A preferable mild acidic solution is a 2% solution of sodium metabisulfite.

In yet another embodiment of the present invention 3,5-bis(cyanoprop-2-yl)benzylbromide may be crystallized from an organic solvent selected from the group consisting of C₁-C₆ alcohols, wherein preferred alcohols are: methanol, ethanol, n-propanol, isopropyl alcohol, n-butanol, and sec-butanol, R₁COOR₂ esters while R₁═C₁-C₅ alkyl and R₂═C₁-C₅ alkyl, wherein preferred esters are: ethyl acetate, n-propyl acetate, isopropyl acetate, n-butyl acetate, and isobutyl acetate, C₁-C₆ ketones, wherein preferred ketones are: acetone, methylethyl ketone, diethyl ketone, methylpropyl ketone, and methylisobutyl ketone, THF, and acctonitrile. More preferably, the solvent for crystallization is selected from the group consisting of isopropyl acetate, ethanol, ethyl acetate or mixture thereof.

In yet another embodiment of the present invention 3,5-bis(2-cyanoprop-2-yl)benzylbromide may be also purified by precipitation from a mixture of a polar solvent and a hydrophobic solvent, wherein preferable polar solvent is dichloromethane and the hydrophobic solvent is a C₅-C₁₀ hydrocarbon, or a mixture of C₅-C₁₀ hydrocarbons. Preferred solvent combinations are mixtures of dichloromethane with heptane or with cyclohexane.

Table 4 provides the results obtained in purifying 3,5-bis(cyanoprop2-yl)benzyl bromide by crystallization or precipitation.

TABLE 4

| Solvent or solvent system | Purification technique | Purity % | Yield % |
|---|---|---|---|
| Ethanol | crystallization | 97.1 | 70 |
| Isopropyl acetate | crystallization | 95.2 | 54 |
| Ethyl acetate | crystallization | 86.2 | 57 |
| Mixture of dichloromethane | precipitation by anti- | 87.5 | 79 |
| and cyclohexane | solvent | | |
| Mixture of dichloromethane | Precipitation by anti- | 88.1 | 78 |
| and heptane | solvent | | |

In yet another embodiment of the present invention the preparation of anastrozole by using isolated and purified 3,5-bis(cyanoprop-2-yl)benzyl bromide, having a purity greater than 97%, thereby enables obtaining anastrozole in high purity and yield.

The following is a summary of the advantages of the process for preparing anastrozole:

1. The present invention provides a process for preparing anastrozole, either free or as a salt form, using a convenient organic solvent, belonging to class 3 or 2, thus avoiding using CCl₄ The preferable solvents used for alkylating 3,5-bis(2-cyanoprop-2-yl)benzylbromide are DMF or isopropyl alcohol or a mixture of DMF and isopropyl alcohol, while the preferable solvents used for brominating 3,5-bis(2-cyanoprop-2-yl)toluene are acetonitrile or dichloromethane;

2. The present invention enables also avoiding the use of the hazardous and toxic reagent benzoyl peroxide; and

3. The intermediate 3,5-bis(cyanoprop-2-yl)benzylbromide may be purified by crystallization or precipitation hence enables obtaining anastrozole in high purity, without the need to use liquid chromatography.

Although, the following examples illustrate the practice of the present invention in some of its embodiments, the examples should not be construed as limiting the scope of the invention. Other embodiments will be apparent to one skilled in the art from consideration of the specification and examples. It is intended that the specification, including the examples, is considered exemplary only, with the scope and spirit of the invention being indicated by the claims which follow.

### EXAMPLES

HPLC measurements of anastrozole samples were performed using HPLC JASCO, LC-1500 series, equipped with Phenomenex Luna Phenyl Hexyl column, 5 μm, 250×4.6 mm, and a UV detector operated on 220 nm. Analyses were performed using the following mobile phase, at flow rate of 1.0 ml/minute, run time 50 minutes.

Solution A: perchloric acid buffer, pH 2.0 (65%), acetonitrile (35%).

Solution B: perchloric acid buffer, pH 2.0 (50%), acetonitrile (50%).

Gradient: t=0 100% A, t=14 100% A, t=14.1 100% B, t=45 100% B, t=45.1 100% A, t=50 100% B.

HPLC measurements of 3,5-bis(2-cyanoprop-2-yl)benzylbromide samples were perfonned using Phenomenex Luna Phenyl Hexyl column, 5 μm, 250×4.6 mm with UV detector operated on 220 nm, temperature of 40° C. and the following mobile phase, at flow rate of 1.0 ml/minute, run time 40 minutes:

Solution A: acetonitrile, Solution B: water.

Gradient: t=0 55% A 45% B, t=25 80% A 20% B. t=30 80% A 20% B, t=30.1 55% A 45% B, t=40 55% A 45% B.

### Example 1

### 2,2′-[5-(1H-1,2,4-triazol-1-ylmethyl)-1,3-phenylene]di(2-methylpropionitrile)

3,5-bis(2-cyanoprop-2-yl)benzylbromide (7.14 g, 0.023 mole) was dissolved in DMF (150 ml), then 1,2,4-triazole sodium salt (2.4 g, 0.028 mole) and potassium carbonate (7 g, 0.051 mol) were added therein. The reaction was stirred at room temperature overnight.

Toluene (200 ml), followed by water (200 ml), were added to the reaction mixture and the two layers were separated. The organic phase was washed with water (3×50 ml) and then with saturated sodium chloride solution (200 ml), then it was acidified with 32% HCl concentrated solution (4.5 ml, 2 eq) until white crystals were obtained. The crystals were filtered off and washed with toluene. Anastrozole hydrochloride was obtained as a white-yellowish solid (6.4 g) in 84% yield.

The hydrochloride salt was converted to the base form by treatment with concentrated sodium carbonate solution (40 ml), followed by extraction of free anastrozole with toluene (2×50 ml). The organic layers were combined and washed with water (50 ml) and dried over magnesium sulfate, concentrated and cooled. The final product was isolated by filtration as white crystals. Free anastrozole was obtained (4.65 g) in 69.5% yield.

### Example 2

### 2,2′-[5-(1H-1,2,4-triazol-1-ylmethyl)-1,3-phenylene]di(2-methylpropionitrile)

3,5-bis(2-cyanoprop-2-yl)benzylbromide (1.4 g, 0.0046 mmol) was dissolved in DMF (30 ml), 1,2,4-triazole sodium salt (0.5 g, 5.4 mmol) and potassium carbonate (1.4 g, 10 mmol) were added therein. The reaction was stirred overnight at room temperature.

Toluene (40 ml), followed by water (40 ml), were added to the reaction mixture and the two layers were separated. The organic phase was washed with water (3×15 ml) and acidified with 48% HBr concentrated solution (1 ml, 2 eq) until white crystals were obtained. The crystals were filtered off and washed with toluene.

The hydrobromide salt was converted to the base form by treatment with concentrated sodium carbonate solution (40 ml), followed by extraction of free anastrozole with toluene (2×50 ml). The organic layers were combined and washed with water (50 ml) and dried over magnesium sulfate, concentrated and cooled. Free anastrozole was obtained by filtration as white crystals (1 g) in 74.7% yield.

### Example 3

### 2,2′-[5-(1H-1,2,4-triazol-1-ylmethyl)-1,3-phenylene]di(2-methylpropionitrile)

3,5-bis(2-cyanoprop-2-yl)benzylbromide (14 g, 0.046 mole) was dissolved in DMF (200 ml), 1,2,4-triazole sodium salt (3.48 g, 0.037 mole) and potassium carbonate (6.97 g, 0.050 mole) were added therein The reaction was stirred for 4 hours at room temperature. After reaction completion (as determined by complete disappearance of 3,5-bis(2-cyanoprop-2-yl)benzylbromide by HPLC), DMF was evaporated under reduced pressure (80% of the original volume of DMF) to obtain an oily residue.

Toluene (200 ml) was added and the salts and excess un-reacted triazole were removed by filtration. The toluene solution was washed with 1N aqueous acidic solution of sodium sulfate and sulfuric acid, pH 1.2 (80 ml) and phases were separated. The process was repeated additional two times.

Part of the solvent was then distilled out under reduced pressure and the remaining toluene solution was saturated with gaseous HCl, which was bubbled into the solution for a period of about 15 minutes until white crystals were obtained. The crystals were filtered off and washed with toluene. The anastrozole hydrochloride salt was suspended in toluene (120 ml) and filtered. Ethyl acetate (120 ml) was added followed by water (100 ml) and sodium carbonate (68 g) and the mixture was stirred for 15 minutes. The phases were separated and the organic phase was washed with water (100 ml). Cyclohexane (3.5 volumes) was added and white crystals were obtained. Free anastrozole was obtained by filtration as white crystals (4.39 g) in 50% yield. Purity: 99.5% by HPLC (content of isoanastrozole: <0.1%).

### Example 4

### 2,2′-[5-(1II-1,2,4-triazol-1-ylmethyl)-1,3-phenylene]di(2-methylpropionitrile)

3,5-bis(2-cyanoprop-2-yl)benzylbromide (100 g, 0.322 mole) was dissolved in a mixture of DMF (150 ml) and isopropyl alcohol (850 ml) and 1,2,4-triazole (25 g, 0.362 mole) and potassium carbonate (50 g, 0.357 mole) were added therein. The reaction was stirred for 20 hours at room temperature under nitrogen. After reaction completion (as determined by complete disappearance of 3,5-bis(2-cyanoprop-2-yl)benzylbromide by HPLC), the solvent was evaporated under reduced pressure (about 80% of the original volume) to obtain an oily residue.

Toluene (1000 ml) was added followed by 1N aqueous acidic solution of sodium sulfate and sulfuric acid, pH 1.2 (500 ml) and stirring was applied for about 10 minutes. The phases were separated and the aqueous phase was extracted with toluene (400 ml). The phases were separated and the two organic layers were combined, washed with 1N aqueous acidic solution of sodium sulfate and sulfuric acid, pH 1.2 (350 ml) and again the phases were separated. The organic layer was washed with 1N aqueous acidic solution of sodium sulfate and sulfuric acid, pH 1.2 (250 ml) and again phases were separated. A sample was withdrawn from the organic layer and injected to HPLC. The content of isoanastrozole was less than 0.05%.

Part of the solvent was then distilled out under reduced pressure and the remaining toluene solution was saturated with gaseous HCl, which was bubbled into the solution for a period of about 1.5 hr at a rate of 20 g/hr and then at a rate of 5 g/hr for about half an hour. The crystals were filtered off and washed with toluene. The anastrozole hydrochloride salt was washed first with toluene (100 ml) and then 3 times with ethyl acetate (800 ml) and filtered to obtain 75 g product in 70.7% yield.

Ethyl acetate (300 ml) was added followed by water (450 ml) and sodium carbonate (50 g) and the mixture was stirred for about 20 minutes. The phases were separated and the organic phase was washed with water (225 ml). Cyclohexane (300 ml) was added and white crystals were obtained. Free anastrozole was obtained having purity of 99.7% by HPLC (content of isoanastrozole: <0.05%).

### Example 5

### 2,2′-[5-(1H-1,2,4-triazol-1-ylmethyl)-1,3-phenylene]di(2-methylpropionitrile)

3,5-bis(2-cyanoprop-2-yl)benzylbromide (10 g, 0.032 mole) was dissolved in isopropyl alcohol (150 ml), and 1,2,4-triazole (2.5 g, 0.036 mole) and lithium tert-butoxide (3.05 g, 0.038 mole) were added therein The reaction was stirred for 12 hours at room temperature under nitrogen. After reaction completion (as determined by complete disappearance of 3,5-bis(2-cyanoprop-2-yl)benzylbromide by HPLC), the solvent was evaporated under reduced pressure and a solid was obtained.

Toluene (30 ml) was added to the solid and reflux was applied for two hours, after which time the solution was immediately filtered and the solvent was evaporated under reduced pressure to obtain a solid. The solid residue, which was obtained on the sinter after the hot filtration, was washed with hot toluene (30 ml), and the solution was added to the solid that was obtained by evaporation. The suspension was refluxed for additional one hour, after which time the solution was evaporated under reduced pressure to obtain crude anastrozole having purity of 99.1% by HPLC.

### Example 6

### Crystallization of crude anastrozole hydrochloride from toluene

Crude anastrozole hydrochloride (3.64 g) was placed in a small glass flask and toluene (12 ml) was added. The flask was heated to 60° C. under vigorous magnetic stirring until complete dissolution has been accomplished and a clear solution was obtained. The heating was discontinued and the temperature was allowed to drop spontaneously to 25° C. The flask was put in ice for 4 hours to effect crystallization. The crystals were collected by filtration and washed with cold toluene (3 ml). Anastrozole hydrochloride (2.61 g) was obtained in 71.7% yield.

### Example 7

### Crystallization of anastrozole hydrochloride from ethanol

Crude anastrozole hydrochloride (0.24 g) was placed in a small glass flask and ethanol (2.2 ml) was added. The flask was heated to 60° C. under vigorous magnetic stirring until complete dissolution has been accomplished and a clear solution was obtained. The heating was discontinued and the temperature was allowed to drop spontaneously to 25° C. The flask was put in ice for 4 hours to effect crystallization. The crystals were collected by filtration and washed with cold ethanol (0.5 ml). Anastrozole hydrochloride (0.18 g) was obtained in 75% yield.

### Example 8

### Crystallization of anastrozole hydrochloride from ethyl acetate

Crude anastrozole hydrochloride (0.24 g) was placed in a small glass flask and ethyl acetate (2.5 ml) was added. The flask was heated to 50° C. under vigorous magnetic stirring until complete dissolution has been accomplished and a clear solution was obtained. The heating was discontinued and the temperature was allowed to drop spontaneously to 25° C. The flask was put in ice for 4 hours to effect crystallization. The crystals were collected by filtration and washed with of cold ethyl acetate (0.8 ml). Anastrozole hydrochloride (0.175 g) was obtained in 73% yield.

### Example 9

### Crystallization of anastrozole hydrochloride from isopropyl alcohol

Crude anastrozole hydrochloride (0.3 g) was placed in a small glass flask and isopropyl alcohol (2 ml) was added. The flask was heated to 50° C. under vigorous magnetic stirring until complete dissolution has been accomplished and a clear solution was obtained. The heating was discontinued and the temperature was allowed to drop spontaneously to 25° C. The flask was put in ice for 4 hours to effect crystallization. The crystals were collected by filtration and washed with cold isopropyl alcohol (0.5 ml). Anastrozole hydrochloride (0.25 g) was obtained in 84% yield.

### Example 10

### Crystallization of anastrozole hydrochloride from a mixture of toluene and isopropyl alcohol

Crude anastrozole hydrochloride (2.62 g) was placed in a small glass flask and mixture of toluene (18 ml) and isopropyl alcohol (13 ml) was added. The flask was heated to 60° C. under vigorous magnetic stirring until complete dissolution has been accomplished and a clear solution was obtained. The heating was discontinued and the temperature was allowed to drop spontaneously to 25° C. The flask was put in ice for 4 hours to effect crystallization. The crystals were collected by filtration and washed with cold mixture of toluene and isopropyl alcohol (8 ml). Anastrozole hydrochloride (2.15 g) was obtained in 82% yield.

### Example 11

### Crystallization of anastrozole hydrochloride from a mixture of acetone and water

Crude anastrozole hydrochloride (0.5 g) was placed in a small glass flask and mixture of acetone (3 ml) and water (1 ml) was added. The flask was heated to 60° C. under vigorous magnetic stirring until complete dissolution has been accomplished and a clear solution was obtained. The heating was discontinued and the temperature was allowed to drop spontaneously to 25° C. The flask was put in ice for 4 hours to effect crystallization. The crystals were collected by filtration and washed with cold mixture of acetone and water (1 ml). Anastrozole hydrochloride (0.3 g) was obtained in 60% yield

### Example 12

### Preparation of 1N aqueous acidic solution of sodium sulfate and sulfuric acid pH 1.2

Sodium sulfate (71 g, 0.5 mole) was dissolved in 0.5 L of deionized water in a 1 L volumetric flask with vigorous stirring, until complete dissolution of the salt. Sulfuric acid 98% (50 g, 0.5 mole) was added dropwise with stirring. Deionized water was added to the mark. Stirring was continued for few minutes to ensure that a uniform mixture was obtained and pH was measured (1.2).

### Example 13

### Preparation of 3,5-bis(2-cyanoprop-2-yl)benzylbromide in dichloromethane without benzoyl peroxide

Into a 500 ml flask equipped with a reflux condenser 3,5-bis(2-cyanoprop-2-yl)toluene (20 g, 0.088 mole) was added and dissolved in dichloromethane (200 ml).

N-bromosuccinimide (15.8 go 0.089 mole) was then added in several portions. UV light was applied (λ=370 nm) with the aid of a UV lamp. The mixture was heated under reflux for 4 hours and then cooled to room temperature. The solids were filtered out, and the solution was washed, first with water (80 ml), followed by NaOH 0.5N (80 ml), 2% solution of sodium metabisulfite (80 ml) and once more with water. The layers were then separated, the organic phase was dried over magnesium sulfate, and the solvent was evaporated under reduced pressure to obtain white crystals (24.6 g, 85% yield). (Purity as determined by HPLC: 87%)

### Example 14

### Preparation of 3,5-bis(2-cyanoprop-2-yl)benzylbromide in dichloromethane in the presence of benzoyl peroxide

Into a 1000 ml flask equipped with a reflux condenser 3,5-bis(2-cyanoprop-2-yl)toluene (40 g, 0.177 mole) was added and dissolved in dichloromethane (400 ml).

N-bromosuccinimide (31.4 g, 0.176 mole) was then added in a number of portions, followed by addition of benzoyl peroxide (0.79 g, 0.003 mole). The mixture was refluxed for 5 hours, additional N-bromo succinimide was added (6.4 g, 0.04 mole), and the reaction mixture was refluxed for further 4 hours. A sample was withdrawn and analysed by HPLC (content of 3,5-bis(2-cyanoprop-2-yl)toluene was less than 2.5%). The reaction mixture was then cooled to room temperature. The solids were filtered out and the solution was washed first with water (150 ml), followed by NaOH 0.5N (150 ml), and a 2% solution of sodium metabisulfite (150 ml).

The layers were separated, the organic phase was dried over magnesium sulfate, and the solvent was evaporated under reduced pressure to obtain white crystals (38.4 g, 0.126 mole, 71.2% yield).

### Example 15

### Preparation of 3,5-bis(2-cyanoprop-2-yl)benzylbromide in acetone

Into a 100 ml flask equipped with a reflux condenser, 3,5-bis(2-cyanoprop-2-yl)toluene (2 g, 0.0088 mole) was added and dissolved in acetone (20 ml).

N-bromosuccinimide (1.92 g, 0.0108 mole) was then added as a single portion, followed by addition of benzoyl peroxide (0.2 g, 0.0008 mole). The mixture was heated under reflux for 4 hours and then cooled to room temperature. The solids were filtered out, and the solution was washed first with water (10 ml), followed by NaOH 0.5N (10 ml) and a 2% solution of sodium metabisulfite (10 ml).

The layers were separated, the organic phase was dried over magnesium sulfate, and the solvent was evaporated under reduced pressure to obtain the product as a solid. 3,5-bis(2-cyanoprop-2-yl)benzylbromide content according to the HPLC chromatogram was 72%

### Example 16

### Preparation of 3,5-bis(2-cyanoprop-2-yl)benzylbromide in acetonitrile

Into a 100 ml flask equipped with a reflux condenser, 3,5-bis(2-cyanoprop-2-yl)toluene (2 g, 0.0088 mole) was added and dissolved in acetonitrile (20 ml). N-bromo succinimide (1.6 g, 0.09 mole) was then added, the mixture was heated under reflux for 4 hours, and then cooled to room temperature. The solids were filtered out and the solution was washed first with water (10 ml), followed by NaOH 0.5N (10 ml), and a 2% solution of sodium metabisulfite (10 ml).

The layers were separated, the organic phase was dried over magnesium sulfate, and the solvent was evaporated under reduced pressure to obtain the product as a solid. 3,5-bis(2-cyanoprop-2-yl)benzylbromide content according to the HPLC chromatogram was 80.6%.

### Example 17

### Crystallization of 3,5-bis(2-cyanoprop-2-yl)benzylbromide from ethanol

Into a 250 ml flask equipped with a mechanical stirrer and a reflux condenser crude 3,5-bis(2-cyanoprop-2-yl)benzyl bromide (28.6 g) was added and refluxed in ethanol (60 ml) until complete dissolution. The temperature was allowed to cool to room temperature, then the flask was put in ice-cold water for two hours. The crystals were filtered, washed with cold ethanol (10 ml) and dried at 50° C. for 4 hours, to give the product (20 g, 70% yield) (Purity as determined by HPLC: 97.1%).

### Example 18

### Crystallization of 3,5-bis(2-cyanoprop-2-yl)benzylbromide from isopropyl acetate

Into a 250 ml flask equipped with mechanical stirrer and reflux condenser crude 3,5-bis(2-cyanoprop-2-yl)benzyl bromide (52.8 g) was added and refluxed in isopropyl acetate (120 ml) until complete dissolution. The temperature was allowed to cool to room temperature, then the flask was put in ice-cold water for two hours. The crystals were filtered and washed with cold isopropyl acetate (20 ml) and dried at 50° C. for 4 hours to obtain 28.6 g in 54% Yield (Purity as determined by HPLC: 95%).

### Example 19

### Crystallization of 3,5-bis(2-cyanoprop-2-yl)benzylbromide from ethyl acetate

Into a 250 ml flask equipped with a mechanical stirrer and a reflux condenser, crude 3,5-bis(2-cyanoprop-2-yl)benzyl bromide (12.4 g) was added and refluxed in ethyl acetate (15 ml) until complete dissolution. The temperature was allowed to cool to room temperature, then the flask was put in ice-cold water for two hours. The crystals were filtered, washed with cold ethyl acetate (4 ml) and dried at 50° C. for 4 hours, to obtain the product (7.1 g, 57% yield, purity as determined by HPLC: 86.2%).

### Example 20

### Precipitation of 3,5-bis(2-cyanoprop-2-yl)benzylbromide from a mixture of dichloromethane and cyclohexane

Into a 500 ml flask, crude 3,5-bis(2-cyanoprop-2-yl)benzyl bromide (20 g) was added and dissolved in dichloromethane (20 ml), under very gentle warming to 30° C., until a clear solution was obtained. The solution was allowed to cool to room temperature, then cyclohexane was added (100 ml), and the solution was mixed for 4 hours. The precipitate was filtered, washed with cyclohexane (20 ml), and dried at 50° C. for 4 hours to obtain the product (15.8 g, 79% yield, purity as determined by HPLC: 89.1%).

### Example 21

### Precipitation of 3,5-bis(2-cyanoprop-2-yl)benzylbromide from a mixture of dichloromethane and heptane

Into a 500 ml flask, crude 3,5-bis(2-cyanoprop-2-yl)benzylbromide (20 g) was added and dissolved in dichloromethane (20 ml) under very gentle warming to 30° C., until a clear solution was obtained. The solution was allowed to cool to room temperature, then heptane was added (100 ml), and the solution was mixed for 4 hours. The precipitate was filtered, washed with heptane (20 ml), and dried at 50° C. for 4 hours to obtain the product (15.6 g, 78% yield, purity as determined by HPLC: 88%)

What is claimed is:

1. Anastrozole in a purity equal to or greater than 99.7%.

2. The anastrozole of claim 1 is further characterized by having a content of isoanastrozole of the following formula: 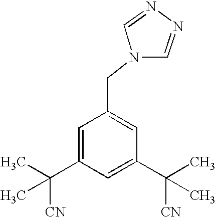
of less than 0.1%, preferably less than 0.05% (according to HPLC).

3. A process for obtaining the pure anastrozole of claim 1, comprising the steps of:
a) dissolving impure anastrozole in a suitable organic solvent;
b) adding aqueous acidic solution having a pH in a range of 0.7-1.7, mixing, selectively extracting and subsequently re-extracting and phase separating;
c) acidifying the organic phase and obtaining anastrozole salt as crystals thereof.
d) filtering the crystals off and washing with the said organic solvent;
e) optionally suspending the crystals in a second organic solvent and converting said anastrozole salt to anastrozole base;
f) optionally partially evaporating the said second organic solvent; and
g) optionally precipitating the said anastrozole base by adding a suitable hydrophobic organic solvent.

4. The process according to claim 3, wherein the suitable organic solvent is selected from the group consisting of dichloromethane, ethyl acetate, isopropyl acetate, butyl acetate, diethyl ether, diisopropyl ether, methyl tert-butyl ether, xylenes, and toluene.

5. The process according to claim 4, wherein the suitable organic solvent is toluene.

6. The process according to claim 3, wherein the suitable aqueous acidic solution is prepared by mixing an acid and its salt, the salt being in anhydrous form or in hydrated form.

7. The process according to claim 6, wherein the acid is an inorganic acid selected from the group consisting of phosphoric acid, phosphorus acid, sulfuric acid, and sodium hydrogen sulfate.

8. The process according to claim 7, wherein the inorganic acid is sulfuric acid.

9. The process according to claim 6, wherein the salt is selected from the group consisting of monosodium phosphate, monopotassium phosphate, monoammonium phosphate, sodium sulfate, potassium sulfate, magnesium sulfate, and ammonium sulfate.

10. The process according to claim 9, wherein the salt is sodium sulfate.

11. The process according to claim 6, wherein the concentration of the aqueous acidic solution is about 1N.

12. The process according to claim 6, wherein the aqueous acidic solution is an acidic solution having a pH in a range of 0.7-1.7.

13. The process according to claim 12, wherein the aqueous acidic solution is an acidic solution having a pH of about 1.2.

14. The process according to claim 3, wherein the organic phase is acidified with an inorganic acid and anastrozole salt crystals are precipitated thereof.

15. The process according to claim 14, wherein the anastrozole salt crystals are precipitated by acidifying the organic phase with a gaseous inorganic acid selected form hydrogen chloride and hydrogen bromide.

16. The process according to claim 3, wherein the second solvent used for suspending the crystals is ethyl acetate.

17. The process according to claim 3, wherein the suitable hydrophobic organic solvent used for precipitating the anastrozole base is cyclohexane.

18. A process for purifying anastrozole by crystallization comprising the steps of:
a) dissolving crude anastrozole in a suitable organic solvent;
b) adding an inorganic acid to thereby form an anastrozole salt;
c) crystallizing said anastrozole salt, to thereby obtain anastrozole salt crystals;
d) filtering off the said crystals and washing with the said organic solvent; and
e) optionally re-crystallizing the anastrozole salt from an organic solvent.

19. The process according to claim 18, wherein the organic solvent is selected from the group consisting of ethyl acetate, isopropyl acetate, n-butyl acetate, isobutyl acetate, dichloromethane, acetone, methylethyl ketone, diethyl ketone, methylpropyl ketone, methylisobutyl ketone, diethyl ether, diisopropyl ether, methyl tert-butyl ether, toluene, and xylenes, or mixture thereof.

20. The process according to claim 19, wherein the organic solvent is toluene or ethyl acetate.

21. The process according to claim 18, wherein the inorganic acid is in a gaseous form or in an aqueous solution.

22. The process according to claim 21, wherein the aqueous inorganic acid solution is a 32% hydrochloric acid solution, thus anastrozole hydrochloride is obtained thereof.

23. The process according to claim 21, wherein the aqueous inorganic acid solution is a 48% hydrobronfric acid solution, thus anastrozole hydrobromide is obtained thereof,

24. The process according to claim 18, wherein the anastrozole hydrochloride is crystallized from an organic solvent selected from the group consisting of toluene, ethanol, ethyl acetate, isopropyl alcohol, acetone, and water or mixture thereof.

25. The process according to claim 18, wherein the purified anastrozole salt is converted to the base form by using an inorganic base selected from the group consisting of sodium hydroxide, potassium hydroxide, sodium carbonate, sodium bicarbonate, potassium carbonate, and potassium bicarbonate.

26. The process according to claim 25, wherein the inorganic base is sodium carbonate.

27. A process for preparing anastrozole, comprising the steps of:
a) dissolving 3,5-bis(2-cyanoprop-2-yl)benzylbromide in an organic solvent other than carbon tetrachloride that belong to class 3 or class 2;
b) reacting 3,5-bis(2-cyanoprop-2-yl)benzylbromide with sodium thiazole or triazole under basic conditions; and
c) purifying the obtained anastrozole essentially as described herein.

28. The process according to claim 27, wherein the organic solvent other than carbon tetrachloride, which belongs to class 3 or class 2, is selected from the group consisting of C₁-C₆ alcohols, wherein preferred alcohols are: methanol, ethanol, n-propanol, isopropyl alcohol, n-butanol, sec-butanol, R₁OR₂ ethers while R₁═C₂-C₅ alkyl and R₂═C₂-C₅ alkyl, wherein preferred ethers are: diethyl ether, diisopropyl ether, methyl tert-butyl ether, and THF, acetonitrile, polar solvents.
wherein preferred polar solvents are: DMF, DMA, DMSO, and NMP, or mixture thereof.

29. The process according to claim 28, wherein the solvents are: DMF, isopropyl alcohol and mixture thereof.

30. The process according to claim 27, wherein the alkylation reaction is carried out in an organic solvent using sodium triazole or triazole and a base selected from the group consisting of potassium carbonate and lithium tert-butoxide.

31. A process for preparing 3,5-bis(2-cyanoprop-2-yl)benzylbromide, the process comprising the steps of:
a) dissolving 3,5-bis(2-cyanoprop-2-yl)toluene in an organic solvent, which belongs to class 3 or class 2;
b) adding a brominating agent, optionally followed by adding benzoyl peroxide;
c) heating the mixture obtained in step b) under reflux for at least about 4 hours, then cooling to room temperature;
d) filtering out solids obtained in step c);
e) washing the remaining organic solution obtained in step d) first with water then with an inorganic basic solution and a mild acidic solution;
f) separating the phases formed in step e) and drying the organic phase obtained over magnesium sulfate;
g) evaporating the solvent optionally under reduced pressure, to thereby produce 3,5-bis(2-cyanoprop-2-yl)benzylbromide; and
h) optionally crystallizing 3,5-bis(2-cyanoprop-2-yl)benzylbromide from an organic solvent.

32. The process according to claim 31, wherein a said class 3 solvent is selected from the group consisting of R₁COOR₂ esters while R₁═C₁-C₅ alkyl and R₂═C₁-C₅ alkyl, wherein preferred esters are: methyl acetate, ethyl acetate, n-propyl acetate, isopropyl acetate, n-butyl acetate, isobutyl acetate, and tert-butyl acetate, C₁-C₆ ketones, wherein preferred ketones are: acetone, methylethyl ketone, diethyl ketone, methylpropyl ketone, and methylisobutyl ketone, and a said class 2 solvent is selected from the group consisting of polar solvents, wherein preferred polar solvents are: DMSO, DMF, DMA, and NMP, halogenated solvents wherein preferred halogenated solvent is dichloromethane, THF, acetonitrile, and isopropyl acetoacetate or mixture thereof.

33. The process according to claim 32, wherein the solvents are: acetonitrile and dichloromethane.

34. The process according to claim 31, wherein the brominating agent is selected from the group consisting of N-bromo succinimide, 1,3-dibromo-5,5-dimethylhydantoin, N-bromoacetamide, bromotrimethylsilane, sodium bromate, and cupric bromide.

35. The process according to claim 34, wherein the brominating agent is N-bromo succinimide.

36. The process according to claim 31, wherein the inorganic basic solution is selected from the group consisting of sodium carbonate, potassium carbonate, potassium hydroxide and sodium hydroxide solutions.

37. The process according to claim 36, wherein the inorganic basic solution is a sodium hydroxide aqueous solution.

38. The process according to claim 31, wherein the mild acidic solution is a 2% solution of sodium metabisulfite.

39. The process according to claim 33, being devoid of using benzoyl peroxide and further comprising applying a UV light following step b), wherein the solvent is dichloromethane.

40. The process according to claim 33, being devoid of using benzoyl peroxide, wherein the solvent is acetonitrile.

41. The process according to claim 31, wherein the reaction solvent is selected from the group consisting of ethyl acetate, isopropyl acetate, acetone, tert-butyl acetate, ethanol or mixture thereof.

42. The process according to claim 31, wherein the 3,5-bis(2-cyanoprop-2-yl)-benzylbromide is crystallized from an organic solvent selected from the group consisting of acetonitrile, C₁-C₆ alcohols, wherein preferred alcohols are: methanol, ethanol, n-propanol, isopropanol, n-butanol, and sec-butanol, R₁COOR₂ esters while R₁═C₁-C₅ alkyl and R₂═C₁-C₅ alkyl, wherein preferred esters are: ethyl acetate, n-propyl acetate, isopropyl acetate, n-butyl acetate, and isobutyl acetate, R₁OR₂ ethers while R₁═C₂-C₅ alkyl and R₂═C₂-C₅ alkyl, wherein preferred ethers are: diethyl ether, diisopropyl ether, methyl tert-butyl ether, and THF or mixture thereof.

43. The process according to claim 42, wherein the suitable organic solvents are: ethyl acetate, isopropyl acetate, ethanol, or mixture thereof.

44. The process according to claim 31, wherein the 3,5-bis(2-cyanoprop-2-yl)benzylbromide is precipitated from a mixture of dichloromethane and a hydrophobic solvent, wherein said hydrophobic solvent is a C₅-C₁₀ hydrocarbon or a mixture of C₅-C₁₀ hydrocarbons.

45. The process according to claim 44, wherein the suitable hydrophobic solvent are cyclohexane and heptane.
